(19) United States
(12) Patent Application Publication
(10) Pub. No.: US 2026/0325859 A1
(43) Pub. Date: Oct. 22, 2026

(54) SYSTEM AND METHOD OF FIBROMYALGIA TREATMENT USING PERIPHERAL MAGNETIC STIMULATION

(71) Applicant: FaraMed Tech Inc., Etobicoke (CA)

(72) Inventors: James Shiraz KHAN, Toronto (CA); Vivek SAINI, Mississauga (CA)

(21) Appl. No.: 19/648,492

(22) Filed: Apr. 15, 2026

Related U.S. Application Data
(60) Provisional application No. 63/790,646, filed on Apr. 17, 2025.

(51) Int. Cl. A61N 2/02; A61N 2/00

(52) U.S. Cl. CPC A61N 2/02; A61N 2/006; A61N 2/008

(57) ABSTRACT

A system and method of peripheral magnetic stimulation for the treatment of fibromyalgia symptoms. The system comprises a pillow applicator having at least two electromagnetic coils embedded within a pillow body formed of non-magnetic or low magnetic permeability material. The pillow body defines a head-receiving region with a locating feature configured to align a patient's head such that the electromagnetic coils are positioned adjacent to the patient's greater occipital nerves. A control unit delivers pulsed electrical energy to the electromagnetic coils to generate pulsed magnetic fields that stimulate the occipital nerves, producing neuromodulation that alleviates fibromyalgia symptoms. The system may be implemented as a benchtop-controlled system or as a portable wearable system. Passive cooling manages heat within the pillow applicator while active cooling is provided in the control unit. Stimulation parameters including intensity, frequency, and pulse duration are adjustable by the user.

### CROSS REFERENCE TO RELATED APPLICATIONS

This application claims priority to and the benefit of U.S. Provisional Application Serial No. 63/790,646, entitled "SYSTEM AND METHOD OF FIBROMYALGIA TREATMENT USING PERIPHERAL MAGNETIC STIMULATION," filed on Apr. 17, 2025, the disclosure of which is incorporated herein by reference in its entirety.

### BACKGROUND

The embodiments described herein relate to non-invasive pain treatment systems and methods, and more particularly, to a system and method for treating fibromyalgia symptoms using peripheral magnetic stimulation of the occipital nerves.

Fibromyalgia is a complex chronic pain disorder characterized by widespread musculoskeletal pain affecting both sides of the body along the axial skeleton. The condition is further characterized by sleep difficulties, fatigue, cognitive impairment (commonly referred to as "fibro fog"), memory impairment, mood disturbance, and irritable bowel syndrome. With a global prevalence estimated at approximately 2% to 3% of the population, fibromyalgia predominantly affects women over 50 years of age, although it can affect individuals of any age and sex.

Current treatment modalities for fibromyalgia have demonstrated limited efficacy. The primary treatment options include pharmacological drug therapy, including pregabalin, duloxetine, and milnacipran. Unfortunately, these drug options provide limited symptomatic relief and are associated with significant side effects including drowsiness, weight gain, dizziness, and gastrointestinal disturbance, which adversely affect patient compliance. Some patients resort to opioid therapy, which carries substantial risks associated with addiction, tolerance, dependence, and accidental overdose. There are currently no surgical or interventional (injection-based) treatment options available for fibromyalgia. As a result, treating physicians face considerable uncertainty in managing fibromyalgia patients. Patients are frequently debilitated by their pain and experience significant difficulty with self-care, daily activities, and maintaining employment.

Recent research has produced promising data suggesting that electrical stimulation of the greater occipital nerves, whether through transcranial direct current stimulation (tDCS) or implantable electrodes, can improve the symptoms of fibromyalgia. However, transcranial direct current stimulation requires precise electrode placement on the scalp and can cause skin irritation, while implantable electrode approaches are invasive, requiring surgical implantation with associated risks of infection, lead migration, and hardware failure.

Peripheral magnetic stimulation (PMS) represents a non-invasive alternative that enables stimulation of peripheral nerves using time-varying magnetic fields rather than direct electrical energy. Unlike electrical stimulation, magnetic fields penetrate tissue without attenuation and without causing discomfort or skin irritation at the surface. However, existing PMS systems are typically designed for clinical use by trained practitioners, are bulky, and are not configured for patient self-administration or for targeting the specific anatomical regions relevant to fibromyalgia treatment.

There exists a pressing need to develop novel, non-invasive, patient-friendly treatment solutions for those suffering from fibromyalgia that can deliver targeted peripheral magnetic stimulation to the occipital nerves in a comfortable, repeatable, and controlled manner.

### BRIEF DESCRIPTION OF THE DRAWINGS

FIGS. 1A and 1B are perspective views of an exemplary fibromyalgia treatment pillow.

FIGS. 2A and 2B are perspective views of the fibromyalgia treatment pillow with electromagnetic coils shown.

FIGS. 3A and 3B are perspective views of a patient lying on the fibromyalgia treatment pillow.

FIGS. 4A and 4B are perspective views of a patient lying on the fibromyalgia treatment pillow with electromagnetic coils shown.

FIG. 5 is a block diagram of an exemplary benchtop peripheral magnetic stimulation system.

FIG. 6 is a block diagram of an exemplary portable wearable peripheral magnetic stimulation system.

FIG. 7 is a diagram illustrating electromagnetic coil locations configured to align with the occipital nerves.

### SUMMARY

In an aspect, a system for treating fibromyalgia symptoms using peripheral magnetic stimulation is disclosed. The system comprises a pillow applicator having a pillow body formed of a non-magnetic or low magnetic permeability material and defining a head-receiving region configured to receive and position a patient's head. At least two electromagnetic coils are embedded within the pillow body and are positioned to generate magnetic fields directed at a greater occipital nerve on each side of the patient's head when the patient's head is received in the head-receiving region. A control unit is coupled to the at least two electromagnetic coils and is configured to deliver electrical energy to the at least two electromagnetic coils to generate pulsed magnetic fields for peripheral magnetic stimulation of the greater occipital nerves, thereby providing neuromodulation for the treatment of fibromyalgia symptoms.

In another aspect, the pillow body defines a locating feature configured to align the patient's head such that the at least two electromagnetic coils are substantially aligned with the patient's greater occipital nerves.

In another aspect, a method of treating fibromyalgia symptoms using peripheral magnetic stimulation is disclosed. The method comprises positioning a patient's head on a pillow applicator having at least two electromagnetic coils embedded therein, aligning the patient's head relative to the at least two electromagnetic coils such that each coil is substantially positioned adjacent to a respective greater occipital nerve of the patient, and delivering pulsed electrical energy to the at least two electromagnetic coils to generate pulsed magnetic fields that stimulate the greater occipital nerves, thereby producing neuromodulation for the treatment of fibromyalgia symptoms.

In another aspect, a portable system for treating fibromyalgia symptoms using peripheral magnetic stimulation is disclosed. The portable system comprises a wearable applicator configured to be worn on a patient's head, at least two electromagnetic coils supported by the wearable applicator and positioned to generate magnetic fields directed at occipital nerves of the patient, and a portable control unit operatively coupled to the at least two electromagnetic coils and comprising a battery, a microcontroller, and a power stage configured to deliver pulsed electrical energy to the at least two electromagnetic coils.

### DETAILED DESCRIPTION

The following detailed description is provided to assist those skilled in the art in practicing the present disclosure. Even so, this detailed description should not be construed to unduly limit the present disclosure, as modifications and variations in the embodiments discussed herein may be made by those of ordinary skill in the art without departing from the spirit or scope of the present inventive discovery.

The contents of each reference cited herein, including the references cited in the Appendix, are hereby incorporated by reference in their entirety.

As used herein, the term "fibromyalgia treatment" encompasses the alleviation, reduction, improvement, or management of one or more symptoms associated with fibromyalgia, including but not limited to widespread pain, fatigue, sleep disturbance, cognitive impairment, and mood disturbance.

As used herein, the term "peripheral magnetic stimulation" or "PMS" refers to the application of time-varying magnetic fields to peripheral nerve structures to induce electrical currents in neural tissue, thereby achieving neuromodulation without requiring direct electrical contact with the body.

As used herein, the term "occipital nerves" includes the greater occipital nerve, the lesser occipital nerve, and the third occipital nerve, unless otherwise specified.

### System Overview

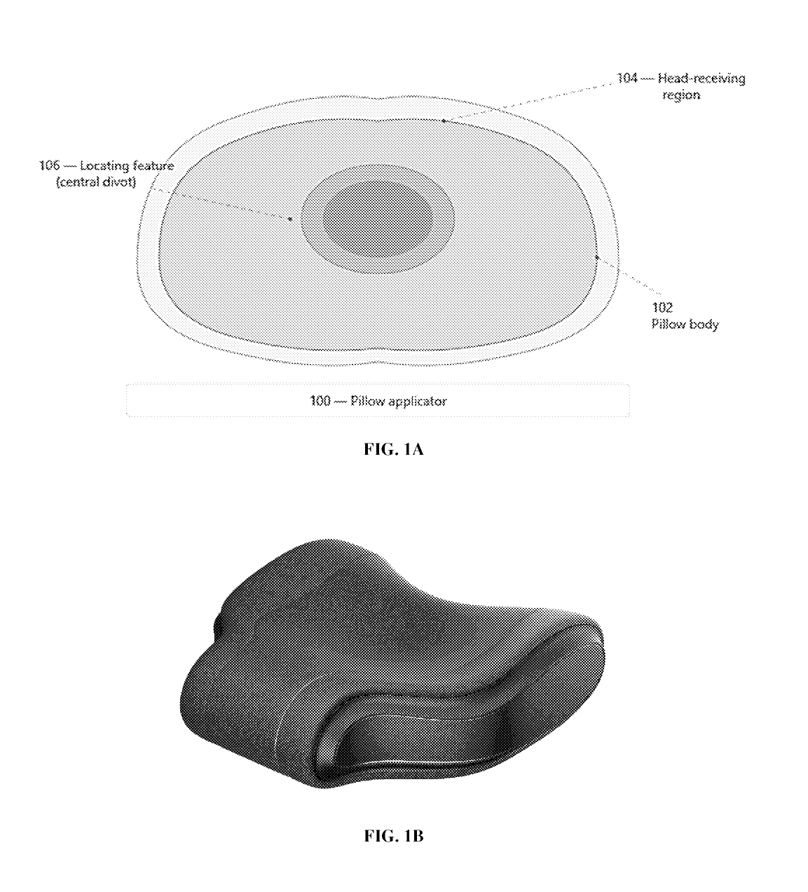

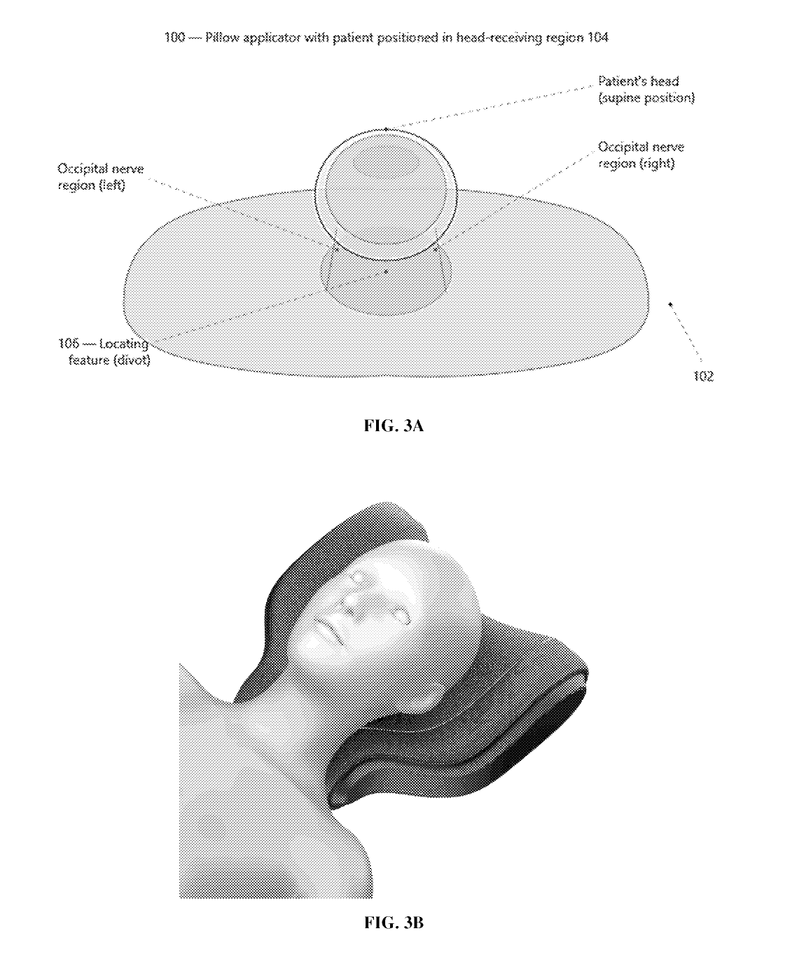

Referring generally to FIGS. 1 through 7, a peripheral magnetic stimulation (PMS) system for the treatment of fibromyalgia symptoms is disclosed. The system is configured to non-invasively stimulate the occipital nerves using pulsed magnetic fields delivered through a pillow applicator or, in alternative embodiments, a wearable applicator. When a patient positions their head on or within the applicator, electromagnetic coils embedded therein generate magnetic fields that penetrate the patient's tissue and induce electrical currents in the occipital nerve fibers, producing neuromodulation that alleviates or improves fibromyalgia symptoms.

The system may be implemented in at least two configurations: (a) a benchtop-controlled system in which a benchtop control unit is connected via a wired connection to a pillow applicator, and (b) a portable wearable system in which a battery-powered portable control unit is coupled to a wearable applicator. Each configuration is described in detail below.

### Pillow Applicator

Referring to FIGS. 1A through 4, the pillow applicator 100 comprises a pillow body 102 formed of one or more non-magnetic or low magnetic permeability materials. Suitable materials for the pillow body 102 include, but are not limited to, cotton, polyester, memory foam, polyurethane foam, latex foam, gel-infused foam, and combinations thereof. The selection of non-magnetic or low magnetic permeability materials is important so as not to interfere with, attenuate, or distort the magnetic fields generated by the electromagnetic coils embedded therein.

The pillow body 102 defines a head-receiving region 104 configured to receive and support a patient's head in a supine position. In some embodiments, the head-receiving region 104 includes a locating feature 106, such as a central divot, depression, contour, or cradle, that is shaped and dimensioned to naturally guide and align the patient's head into a predetermined position. The locating feature 106 is configured such that when the patient's head is received therein, the patient's occipital nerves are substantially aligned with the electromagnetic coils embedded within the pillow body 102. The locating feature 106 is designed to accommodate a range of head sizes across most of the adult population.

The pillow applicator 100 may have a range of dimensions configured to accommodate the patient's head and to provide sufficient surface area for coil placement. In some embodiments, the pillow body 102 has a length in the range of approximately 16 inches to approximately 24 inches, a width in the range of approximately 12 inches to approximately 20 inches, and a height or thickness in the range of approximately 3 inches to approximately 7 inches.

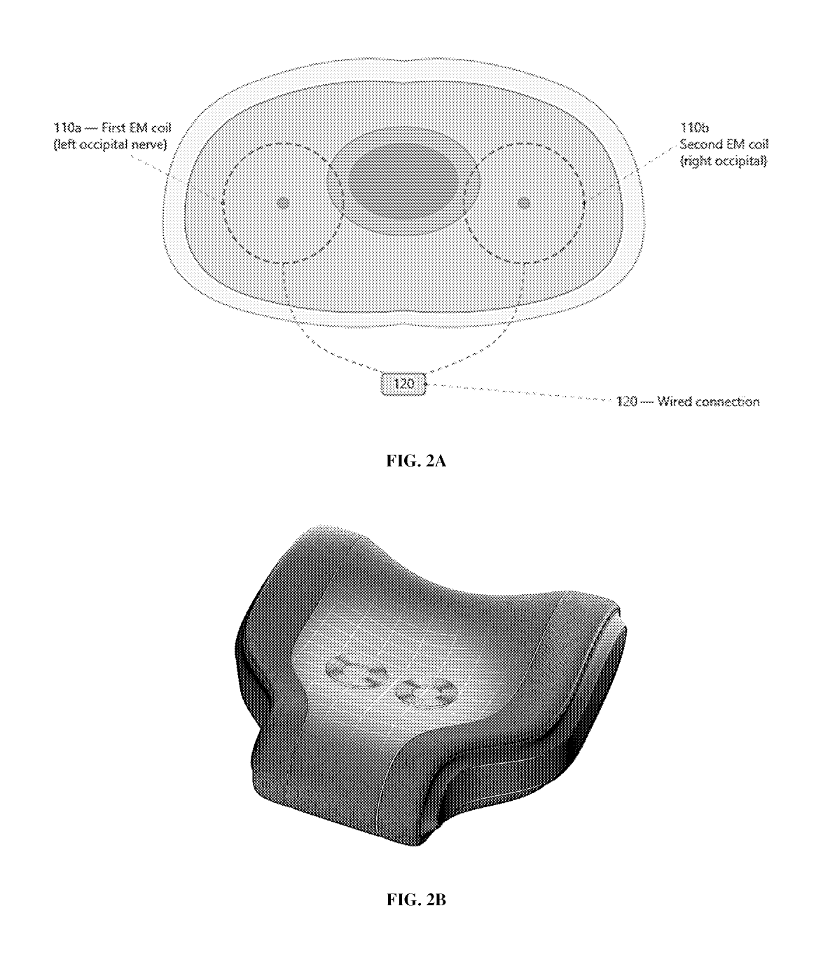

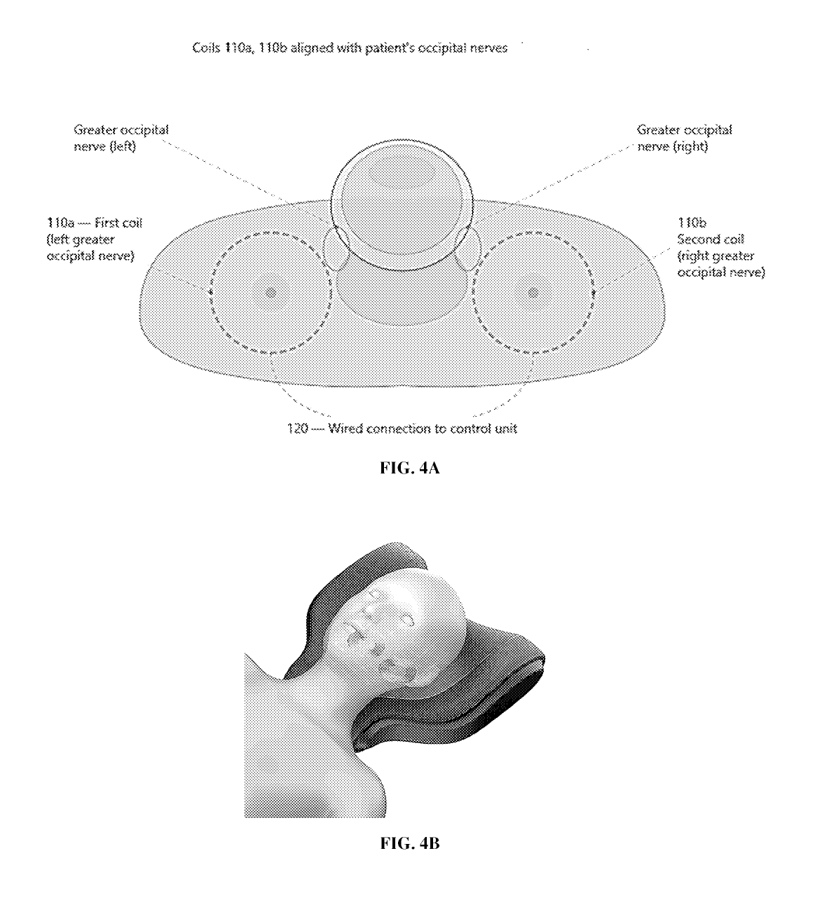

Referring to FIGS. 2A and 4A, at least two electromagnetic coils 110a, 110b are embedded within the pillow body 102. The electromagnetic coils 110a, 110b are positioned on opposing sides of the head-receiving region 104 such that, when a patient's head is positioned in the head-receiving region 104, a first coil 110a is substantially aligned with the patient's greater occipital nerve on a first side (e.g., left side) of the patient's head, and a second coil 110b is substantially aligned with the patient's greater occipital nerve on a second side (e.g., right side) of the patient's head.

In some embodiments, the electromagnetic coils 110a, 110b are circular coils. In other embodiments, the coils may be figure-eight coils, D-shaped coils, or other coil geometries known in the art for peripheral magnetic stimulation. In embodiments employing two or more coils, the coils are arranged and configured so as not to interfere with each other. For example, the coils may be arranged in a figure-eight configuration such that the magnetic fields generated by adjacent coils do not destructively interfere. In some embodiments, the coils are configured to independently stimulate the left and right greater occipital nerves, respectively.

The electromagnetic coils 110a, 110b are positioned close to the surface of the pillow body 102 that contacts the patient's head, thereby maximizing the magnetic field strength at the target nerve location. In some embodiments, the coils are positioned within approximately 0.25 inches to approximately 1.5 inches from the patient-contacting surface of the pillow body 102.

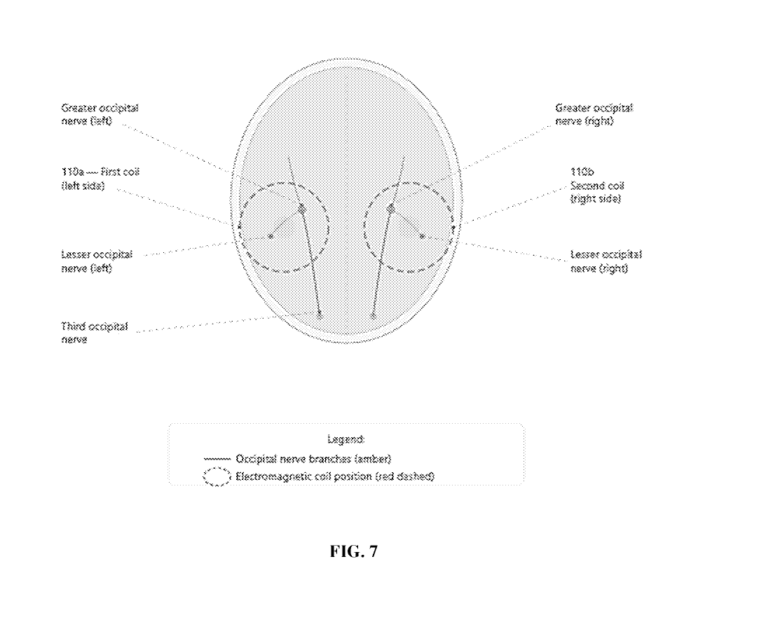

Referring to FIG. 7, the electromagnetic coils 110a, 110b are positioned to align with the occipital nerve region, including one or more of the greater occipital nerve, the lesser occipital nerve, and the third occipital nerve. In some embodiments, the coils are primarily positioned to target the greater occipital nerve on each side of the patient's head. In other embodiments, additional coils may be included to target the lesser occipital nerve and/or the third occipital nerve.

The pillow applicator 100 further comprises a wired connection 120 extending from the pillow body 102 for coupling the electromagnetic coils 110a, 110b to the control unit. The wired connection 120 ensures consistent and reliable power delivery and signal communication between the control unit and the coils.

### Pillow Cooling System

The pillow applicator 100 includes a passive cooling system configured to manage heat generated by the electromagnetic coils 110a, 110b during operation. The passive cooling system may comprise heat-spreading materials, such as thermally conductive fabrics, graphene-based layers, copper mesh, aluminum heat spreaders, phase-change materials, or other passive thermal management materials, that are integrated into or adjacent to the electromagnetic coils 110a, 110b within the pillow body 102. The passive cooling system dissipates heat generated by the coils without the need for fans or active cooling components within the pillow, thereby maintaining patient comfort and minimizing noise during treatment.

In some embodiments, the pillow applicator 100 may include one or more temperature sensors 130 positioned adjacent to or in thermal contact with the electromagnetic coils 110a, 110b. The temperature sensors 130 are configured to monitor the temperature of the coils and/or the pillow surface and to communicate temperature data to the control unit. The control unit may be configured to reduce stimulation power, adjust duty cycle, or cease stimulation if the monitored temperature exceeds a predetermined threshold, thereby providing thermal safety protection.

### Benchtop Control Unit

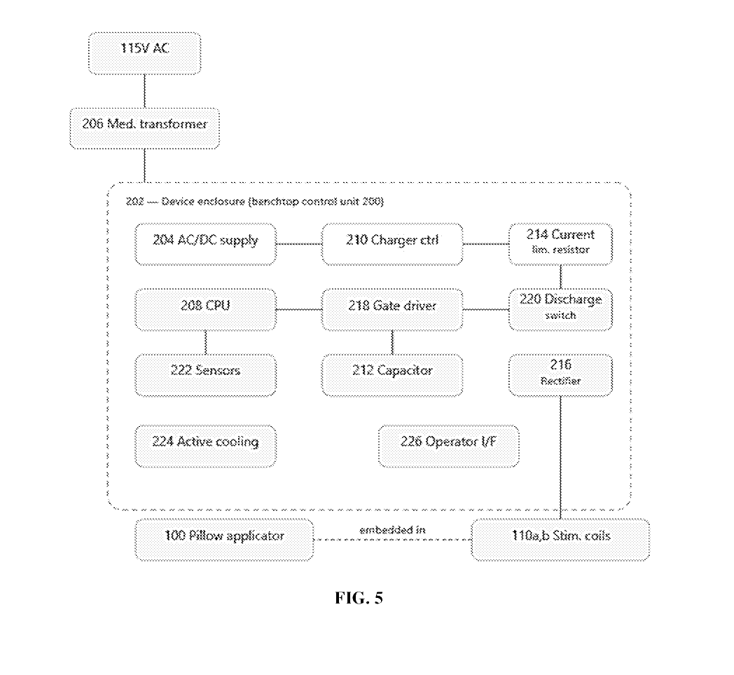

Referring to FIG. 5, in a first configuration, the system comprises a benchtop control unit 200. According to FIG. 5, the benchtop control unit 200 serves as the main power source and pulse controller for the system. The benchtop control unit 200 is housed within a device enclosure 202.

The benchtop control unit 200 comprises an AC/DC power supply 204 configured to receive alternating current (AC) power from a standard electrical outlet. In some embodiments, the AC/DC power supply 204 is configured to receive power from a medical-grade transformer 206 that receives 115V AC (or, in other embodiments, 230V AC for international markets). The AC/DC power supply 204 converts the AC power to direct current (DC) power for use by the internal components of the control unit.

The benchtop control unit 200 further comprises a central processing unit (CPU) 208 or microcontroller configured to control the overall operation of the system. The CPU 208 is programmed to control stimulation parameters including, but not limited to, pulse intensity, pulse frequency, pulse duration, pulse waveform, duty cycle, treatment duration, and ramp-up/ramp-down profiles. In some embodiments, the CPU 208 is configured to execute one or more pre-programmed treatment protocols stored in a memory.

The benchtop control unit 200 further comprises a charger control 210 operatively coupled to a capacitor 212 for energy storage. The charger control 210 is configured to charge the capacitor 212 to a predetermined voltage level. A current-limiting resistor 214 is provided to limit the charging current to the capacitor 212. A rectifier 216 is provided in the charging circuit.

A gate driver 218 is operatively coupled to a high-power discharge switch 220. The CPU 208 controls the gate driver 218 to actuate the high-power discharge switch 220, thereby discharging the energy stored in the capacitor 212 through the electromagnetic coils 110a, 110b to generate a pulsed magnetic field. The timing and pattern of discharge pulses are controlled by the CPU 208 to achieve the desired stimulation parameters.

The benchtop control unit 200 further comprises a plurality of sensors 222 configured to monitor system parameters. The plurality of sensors 222 may include one or more of: coil temperature sensors configured to measure the temperature of the electromagnetic coils, resting temperature sensors configured to measure ambient or enclosure temperature, input voltage sensors configured to monitor the input power supply voltage, and capacitor voltage sensors configured to monitor the charge state of the energy storage capacitor 212.

The benchtop control unit 200 further comprises an active cooling system 224, such as one or more fans, heat sinks, or thermoelectric coolers, configured to maintain the electronics within the device enclosure 202 at a safe operating temperature.

The benchtop control unit 200 further comprises an operator interface 226 for debugging, troubleshooting, and system configuration. In some embodiments, the operator interface 226 comprises a display screen (e.g., LCD or OLED), one or more input controls (e.g., buttons, knobs, touchscreen), and/or indicator lights. In some embodiments, the operator interface 226 is configured to allow the user to adjust stimulation settings including intensity, frequency, and pulse duration.

### Portable Wearable System

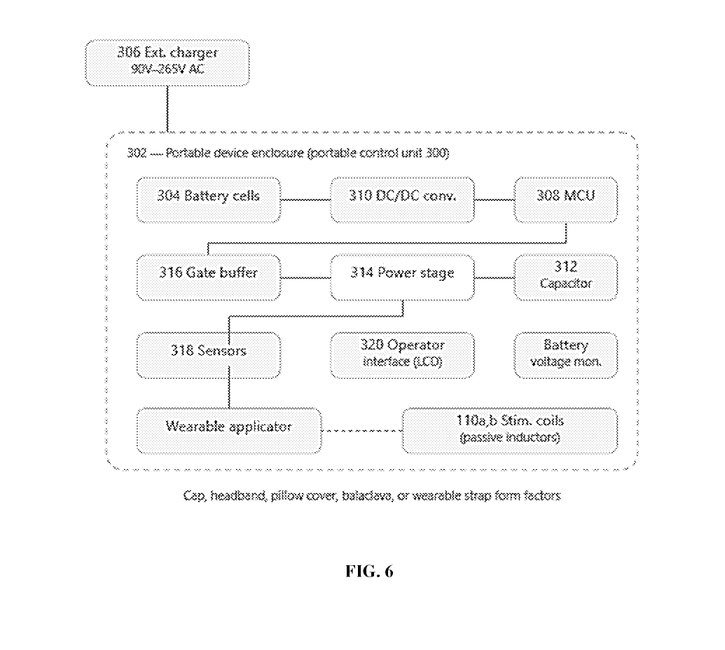

Referring to FIG. 6, in a second configuration, the system comprises a portable control unit 300 configured for use with a wearable applicator. According to FIG. 6, the portable control unit 300 is housed within a portable device enclosure 302 and is configured for battery-powered operation, enabling the patient to receive treatment without being tethered to a wall outlet.

The portable control unit 300 comprises one or more battery cells 304 serving as the primary power source. The battery cells 304 may be rechargeable lithium-ion, lithium-polymer, or other suitable battery chemistries. An external charger 306 is provided for recharging the battery cells 304 and is configured to receive AC input in the range of approximately 90V to approximately 265V AC, thereby supporting both domestic and international power standards.

The portable control unit 300 further comprises a microcontroller 308 configured to control stimulation parameters and system operation. A DC/DC converter 310 is provided to convert the battery voltage to the required operating voltages for the system components. A capacitor 312 is provided for energy storage, and a power stage 314 and gate buffer 316 are provided to drive the discharge of stored energy through the electromagnetic coils.

The portable control unit 300 further comprises one or more sensors 318 configured to monitor system parameters including battery voltage, coil temperature, and other operational parameters. An operator interface 320, such as an LCD screen, display, or LED indicators, is provided for user interaction and system status display.

The portable control unit 300 is operatively coupled to one or more stimulation coils, which may be passive inductors or passive copper coils, housed within the wearable applicator.

In some embodiments, the wearable applicator may take the form of a pillow, a pillow cover, a head cushion, a wearable strap, a headband, a cap, a balaclava, or another form of head covering or accessory configured to position the electromagnetic coils adjacent to the patient's occipital nerves. In some embodiments, the wearable applicator is configured to strap onto or attach to an existing pillow as an accessory.

### Stimulation Parameters and Treatment Protocols

The system is configured to deliver pulsed magnetic stimulation to the occipital nerves with adjustable stimulation parameters. In some embodiments, the adjustable stimulation parameters include one or more of: stimulation intensity (magnetic field strength), stimulation frequency (pulse repetition rate), pulse duration, pulse waveform shape, duty cycle (ratio of stimulation on-time to off-time), total treatment session duration, and ramp-up and ramp-down profiles.

In some embodiments, the system stores one or more pre-programmed treatment protocols optimized for fibromyalgia symptom management. The treatment protocols may specify particular combinations of the above stimulation parameters.

In some embodiments, the system is configured to apply machine learning techniques to aggregate datasets derived from user feedback, user settings, and clinical research data to optimize stimulation parameters for individual patients. The system may prompt the user with questions through the operator interface to learn the user's preferred settings and may intelligently suggest improved settings based on the user's individual data and/or aggregate datasets from research and multiple users.

### Methods of Treatment

In use, the method of treating fibromyalgia symptoms using the disclosed system comprises the following steps. A patient positions their head on the pillow applicator such that the patient's head is received in the head-receiving region. The locating feature of the pillow applicator guides the patient's head into alignment such that the electromagnetic coils are substantially aligned with the patient's greater occipital nerves on each side of the head. The control unit is activated, and the user selects or confirms the desired stimulation parameters through the operator interface. The control unit delivers pulsed electrical energy to the electromagnetic coils, which generate pulsed magnetic fields that penetrate the patient's tissue and stimulate the greater occipital nerves. The stimulation produces neuromodulation of the occipital nerve pathways, resulting in alleviation or improvement of fibromyalgia symptoms including pain, fatigue, sleep disturbance, and cognitive impairment. The treatment session continues for a predetermined duration, after which the control unit ceases stimulation.

In some embodiments, the method further comprises monitoring the temperature of the electromagnetic coils during treatment and automatically adjusting or ceasing stimulation if the coil temperature exceeds a safety threshold.

In some embodiments, the method further comprises recording the stimulation parameters and patient-reported outcomes for the treatment session and using the recorded data, optionally in combination with machine learning analysis, to adjust stimulation parameters for subsequent treatment sessions.

### Alternative Embodiments

In alternative embodiments, the entire system electronics may be embedded within the pillow applicator itself, eliminating the need for a separate benchtop control unit. In such embodiments, the pillow applicator may include an integrated battery, microcontroller, power stage, and energy storage capacitor.

In alternative embodiments, depending on the power requirements and duty cycle of the stimulation, the pillow applicator and/or control unit may not require a dedicated cooling system.

In alternative embodiments, additional electromagnetic coils may be provided within the pillow applicator or wearable applicator to target the lesser occipital nerve and/or the third occipital nerve, in addition to or instead of the greater occipital nerve.

In alternative embodiments, the system may include wireless communication capabilities (e.g., Bluetooth, Wi-Fi) to enable the control unit to communicate with a smartphone application, tablet, or remote monitoring system for treatment tracking, parameter adjustment, and data collection.

According to the disclosure, a system for treating fibromyalgia symptoms using peripheral magnetic stimulation is disclosed. The system comprises a pillow applicator comprising a pillow body formed of a non-magnetic or low magnetic permeability material, the pillow body defining a head-receiving region configured to receive and position a patient's head, at least two electromagnetic coils embedded within the pillow body and positioned on opposing sides of the head-receiving region such that, when the patient's head is received in the head-receiving region, a first electromagnetic coil is substantially aligned with a greater occipital nerve on a first side of the patient's head and a second electromagnetic coil is substantially aligned with a greater occipital nerve on a second side of the patient's head and a control unit operatively coupled to the at least two electromagnetic coils and configured to deliver pulsed electrical energy to the at least two electromagnetic coils to generate pulsed magnetic fields for peripheral magnetic stimulation of the greater occipital nerves, thereby producing neuromodulation for the treatment of fibromyalgia symptoms.

According to the disclosure, the pillow body of the system defines a locating feature in the head-receiving region, the locating feature configured to guide and align the patient's head into a predetermined position such that the patient's greater occipital nerves are substantially aligned with the at least two electromagnetic coils. The locating feature comprises a central divot, depression, or contour in the pillow body.

According to the disclosure, the pillow body of the system is formed from one or more materials selected from the group consisting of: cotton, polyester, memory foam, polyurethane foam, latex foam, and gel-infused foam. Furthermore, the at least two electromagnetic coils are positioned within approximately 0.25 inches to approximately 1.5 inches from a patient-contacting surface of the pillow body.

According to the disclosure, the at least two electromagnetic coils are configured in a figure-eight arrangement such that the magnetic fields generated by adjacent coils do not destructively interfere with one another. Furthermore, the at least two electromagnetic coils are configured to independently stimulate the greater occipital nerve on the first side and the greater occipital nerve on the second side of the patient's head.

According to the disclosure, the system further comprises a passive cooling system integrated within the pillow body and configured to dissipate heat generated by the at least two electromagnetic coils during operation without the use of active cooling components within the pillow applicator. The passive cooling system comprises one or more heat-spreading materials selected from the group consisting of: thermally conductive fabrics, graphene-based layers, copper mesh, aluminum heat spreaders, and phase-change materials.

According to the disclosure, the system further comprises one or more temperature sensors positioned adjacent to the at least two electromagnetic coils and configured to monitor coil temperature, wherein the control unit is configured to adjust or cease stimulation if the monitored temperature exceeds a predetermined threshold.

According to the disclosure, the control unit of the system comprises an energy storage capacitor, a charger control configured to charge the energy storage capacitor to a predetermined voltage level, a gate driver operatively coupled to a high-power discharge switch and a central processing unit configured to control the gate driver to actuate the high-power discharge switch to discharge energy stored in the energy storage capacitor through the at least two electromagnetic coils. Furthermore, the control unit is configured to allow adjustment of one or more stimulation parameters selected from the group consisting of: stimulation intensity, stimulation frequency, pulse duration, pulse waveform, duty cycle, and treatment session duration.

According to the disclosure, the control unit comprises an active cooling system configured to maintain electronics within the control unit at a safe operating temperature. The control unit is a benchtop control unit connected to the pillow applicator via a wired connection.

According to the disclosure, the system further comprises one or more additional electromagnetic coils embedded within the pillow body and positioned to target one or more of the lesser occipital nerve and the third occipital nerve.

According to the disclosure, a portable system for treating fibromyalgia symptoms using peripheral magnetic stimulation is disclosed. The portable system comprises a wearable applicator configured to be positioned on or about a patient's head, the wearable applicator comprising a body formed of a non-magnetic or low magnetic permeability material, at least two electromagnetic coils supported by the wearable applicator and positioned such that, when the wearable applicator is positioned on the patient's head, the at least two electromagnetic coils are substantially aligned with the patient's occipital nerves and a portable control unit operatively coupled to the at least two electromagnetic coils.

According to the disclosure, the portable control unit of the portable system comprises one or more battery cells, a microcontroller configured to control stimulation parameters and a power stage configured to deliver pulsed electrical energy to the at least two electromagnetic coils to generate pulsed magnetic fields for peripheral magnetic stimulation of the occipital nerves, thereby producing neuromodulation for the treatment of fibromyalgia symptoms. The wearable applicator is selected from the group consisting of: a pillow, a pillow cover, a head cushion, a wearable strap, a headband, a cap, and a balaclava.

According to the disclosure, the portable system further comprises an external charger configured to receive AC input in the range of approximately 90V to approximately 265V AC for recharging the one or more battery cells. The portable system further comprises a DC/DC converter configured to convert a voltage of the one or more battery cells to one or more operating voltages for the microcontroller and the power stage.

According to the disclosure, the portable system further comprises an energy storage capacitor, wherein the power stage is configured to discharge energy stored in the energy storage capacitor through the at least two electromagnetic coils. The portable system further comprises an operator interface comprising a display configured to present system status information and to receive user input for adjusting stimulation parameters.

According to the disclosure, the portable system further comprises one or more sensors configured to monitor one or more of battery voltage, coil temperature, and stimulation output parameters. The at least two electromagnetic coils are positioned to independently stimulate the greater occipital nerve on each side of the patient's head.

According to the disclosure, a method of treating fibromyalgia symptoms using peripheral magnetic stimulation is disclosed, the method comprises the steps of positioning a patient's head on a pillow applicator comprising a pillow body formed of a non-magnetic or low magnetic permeability material, the pillow body having at least two electromagnetic coils embedded therein, aligning the patient's head relative to the at least two electromagnetic coils such that a first electromagnetic coil is substantially positioned adjacent to a greater occipital nerve on a first side of the patient's head and a second electromagnetic coil is substantially positioned adjacent to a greater occipital nerve on a second side of the patient's head and delivering pulsed electrical energy to the at least two electromagnetic coils to generate pulsed magnetic fields that stimulate the greater occipital nerves, thereby producing neuromodulation for the treatment of fibromyalgia symptoms.

According to the disclosure, the step of aligning the patient's head of the method comprises guiding the patient's head into a locating feature defined in the pillow body, the locating feature configured to position the patient's head such that the patient's greater occipital nerves are substantially aligned with the at least two electromagnetic coils. The method further comprises adjusting one or more stimulation parameters selected from the group consisting of: stimulation intensity, stimulation frequency, pulse duration, duty cycle, and treatment session duration.

According to the disclosure, the method further comprises monitoring a temperature of the at least two electromagnetic coils during delivery of the pulsed electrical energy and automatically adjusting or ceasing stimulation if the temperature exceeds a predetermined safety threshold. The method further comprises recording stimulation parameters and patient-reported outcomes for a treatment session and using the recorded data to adjust stimulation parameters for a subsequent treatment session.

According to the disclosure, using the recorded data of the method comprises applying machine learning analysis to the recorded data in combination with aggregate data from a plurality of patients to determine optimized stimulation parameters. The pulsed magnetic fields of the method are generated at a stimulation frequency, pulse duration, and intensity that are independently adjustable by a user via an operator interface of a control unit.

According to the disclosure, a pillow applicator for use in a peripheral magnetic stimulation system for treating fibromyalgia symptoms is disclosed. The pillow applicator comprises a pillow body formed of a non-magnetic or low magnetic permeability material, the pillow body defining a head-receiving region having a locating feature configured to receive and align a patient's head, at least two electromagnetic coils embedded within the pillow body and positioned on opposing sides of the locating feature such that, when the patient's head is aligned by the locating feature, the at least two electromagnetic coils are substantially aligned with the patient's greater occipital nerves, a passive cooling system integrated within the pillow body and configured to dissipate heat generated by the at least two electromagnetic coils and a wired connection extending from the pillow body for coupling the at least two electromagnetic coils to a control unit.

According to the disclosure, the locating feature of the pillow applicator comprises a central divot configured to accommodate a range of head sizes across an adult population. The at least two electromagnetic coils are circular coils arranged to independently stimulate the greater occipital nerve on each side of the patient's head.

According to the disclosure, the passive cooling system of the pillow applicator comprises one or more heat-spreading materials positioned adjacent to the at least two electromagnetic coils. The pillow applicator of claim 31, further comprising one or more temperature sensors positioned in thermal contact with the at least two electromagnetic coils and configured to communicate temperature data to the control unit via the wired connection.

Implementations disclosed herein provide systems, methods and apparatus for generating or augmenting training data sets for machine learning training. For example, machine learning training can be used to aggregate datasets from feedback from the users settings. On can further ask questions in the application to learn a user’s preferred settings and intelligently suggest better settings based on the user’s data and also based on aggregate datasets from research and multiple users.

The functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor. A “module” can be considered as a processor executing computer-readable code.

A processor as described herein can be a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, or microcontroller, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, any of the signal processing algorithms described herein may be implemented in analog circuitry. In some embodiments, a processor can be a graphics processing unit (GPU). The parallel processing capabilities of GPUs can reduce the amount of time for training and using neural networks (and other machine learning models) compared to central processing units (CPUs). In some embodiments, a processor can be an ASIC including dedicated machine learning circuitry custom-build for one or both of model training and model inference.

The disclosed or illustrated tasks can be distributed across multiple processors or computing devices of a computer system, including computing devices that are geographically distributed. The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.” While the foregoing written description of the system enables one of ordinary skill to make and use what is considered presently to be the best mode thereof, those of ordinary skill will understand and appreciate the existence of variations, combinations, and equivalents of the specific embodiment, method, and examples herein. The system should therefore not be limited by the above-described embodiment, method, and examples, but by all embodiments and methods within the scope and spirit of the system. Thus, the present disclosure is not intended to be limited to the implementations shown herein but is to be accorded the widest scope consistent with the principles and novel features disclosed herein.

Portions of this application, including the specification and / or claims, may have been drafted using AI-assisted tools under the direction and supervision of a human practitioner. All inventive contributions are attributable to the listed inventors.

What is claimed:

1. A system for treating fibromyalgia symptoms using peripheral magnetic stimulation, the system comprising:
a pillow applicator comprising a pillow body formed of a non-magnetic or low magnetic permeability material, the pillow body defining a head-receiving region configured to receive and position a patient's head;
at least two electromagnetic coils embedded within the pillow body and positioned on opposing sides of the head-receiving region such that, when the patient's head is received in the head-receiving region, a first electromagnetic coil is substantially aligned with a greater occipital nerve on a first side of the patient's head and a second electromagnetic coil is substantially aligned with a greater occipital nerve on a second side of the patient's head; and
a control unit operatively coupled to the at least two electromagnetic coils and configured to deliver pulsed electrical energy to the at least two electromagnetic coils to generate pulsed magnetic fields for peripheral magnetic stimulation of the greater occipital nerves, thereby producing neuromodulation for the treatment of fibromyalgia symptoms.

2. The system of claim 1, wherein the pillow body defines a locating feature in the head-receiving region, the locating feature configured to guide and align the patient's head into a predetermined position such that the patient's greater occipital nerves are substantially aligned with the at least two electromagnetic coils and the locating feature comprises a central divot, depression, or contour in the pillow body.

3. The system of claim 1, wherein the pillow body is formed from one or more materials selected from the group consisting of: cotton, polyester, memory foam, polyurethane foam, latex foam, and gel-infused foam.

4. The system of claim 1, wherein the at least two electromagnetic coils are positioned within approximately 0.25 inches to approximately 1.5 inches from a patient-contacting surface of the pillow body.

5. The system of claim 1, wherein the at least two electromagnetic coils are configured in a figure-eight arrangement such that the magnetic fields generated by adjacent coils do not destructively interfere with one another.

6. The system of claim 1, wherein the at least two electromagnetic coils are configured to independently stimulate the greater occipital nerve on the first side and the greater occipital nerve on the second side of the patient's head.

7. The system of claim 1, further comprising a passive cooling system integrated within the pillow body and configured to dissipate heat generated by the at least two electromagnetic coils during operation without the use of active cooling components within the pillow applicator.

8. The system of claim 7, wherein the passive cooling system comprises one or more heat-spreading materials selected from the group consisting of: thermally conductive fabrics, graphene-based layers, copper mesh, aluminum heat spreaders, and phase-change materials.

9. The system of claim 1, further comprising one or more temperature sensors positioned adjacent to the at least two electromagnetic coils and configured to monitor coil temperature, wherein the control unit is configured to adjust or cease stimulation if the monitored temperature exceeds a predetermined threshold.

10. The system of claim 1, wherein the control unit comprises:
an energy storage capacitor;
a charger control configured to charge the energy storage capacitor to a predetermined voltage level;
a gate driver operatively coupled to a high-power discharge switch; and
a central processing unit configured to control the gate driver to actuate the high-power discharge switch to discharge energy stored in the energy storage capacitor through the at least two electromagnetic coils.

11. The system of claim 1, wherein the control unit is configured to allow adjustment of one or more stimulation parameters selected from the group consisting of: stimulation intensity, stimulation frequency, pulse duration, pulse waveform, duty cycle, and treatment session duration.

12. The system of claim 1, wherein the control unit comprises:
an active cooling system configured to maintain electronics within the control unit at a safe operating temperature;
wherein the control unit is a benchtop control unit connected to the pillow applicator via a wired connection.

13. The system of claim 1, further comprising one or more additional electromagnetic coils embedded within the pillow body and positioned to target one or more of the lesser occipital nerve and the third occipital nerve.

14. A portable system for treating fibromyalgia symptoms using peripheral magnetic stimulation, the portable system comprising:
a wearable applicator configured to be positioned on or about a patient's head, the wearable applicator comprising a body formed of a non-magnetic or low magnetic permeability material;
at least two electromagnetic coils supported by the wearable applicator and positioned such that, when the wearable applicator is positioned on the patient's head, the at least two electromagnetic coils are substantially aligned with the patient's occipital nerves; and
a portable control unit operatively coupled to the at least two electromagnetic coils, the portable control unit comprising:
one or more battery cells;
a microcontroller configured to control stimulation parameters; and
a power stage configured to deliver pulsed electrical energy to the at least two electromagnetic coils to generate pulsed magnetic fields for peripheral magnetic stimulation of the occipital nerves, thereby producing neuromodulation for the treatment of fibromyalgia symptoms.

15. The portable system of claim 14, further comprising an external charger configured to receive AC input in the range of approximately 90V to approximately 265V AC for recharging the one or more battery cells.

16. The portable system of claim 14, further comprising:
a DC/DC converter configured to convert a voltage of the one or more battery cells to one or more operating voltages for the microcontroller and the power stage; and
an energy storage capacitor, wherein the power stage is configured to discharge energy stored in the energy storage capacitor through the at least two electromagnetic coils.

17. A method of treating fibromyalgia symptoms using peripheral magnetic stimulation, the method comprising:
positioning a patient's head on a pillow applicator comprising a pillow body formed of a non-magnetic or low magnetic permeability material, the pillow body having at least two electromagnetic coils embedded therein;
aligning the patient's head relative to the at least two electromagnetic coils such that a first electromagnetic coil is substantially positioned adjacent to a greater occipital nerve on a first side of the patient's head and a second electromagnetic coil is substantially positioned adjacent to a greater occipital nerve on a second side of the patient's head; and
delivering pulsed electrical energy to the at least two electromagnetic coils to generate pulsed magnetic fields that stimulate the greater occipital nerves, thereby producing neuromodulation for the treatment of fibromyalgia symptoms.

18. The method of claim 17, wherein aligning the patient's head comprises guiding the patient's head into a locating feature defined in the pillow body, the locating feature configured to position the patient's head such that the patient's greater occipital nerves are substantially aligned with the at least two electromagnetic coils.

19. The method of claim 17, further comprising:
adjusting one or more stimulation parameters selected from the group consisting of: stimulation intensity, stimulation frequency, pulse duration, duty cycle, and treatment session duration;
monitoring a temperature of the at least two electromagnetic coils during delivery of the pulsed electrical energy and automatically adjusting or ceasing stimulation if the temperature exceeds a predetermined safety threshold; and
recording stimulation parameters and patient-reported outcomes for a treatment session and using the recorded data to adjust stimulation parameters for a subsequent treatment session.

20. The method of claim 17, wherein
using the recorded data comprises applying machine learning analysis to the recorded data in combination with aggregate data from a plurality of patients to determine optimized stimulation parameters; and
the pulsed magnetic fields are generated at a stimulation frequency, pulse duration, and intensity that are independently adjustable by a user via an operator interface of a control unit.
